(19) United States
(12) Patent Application Publication
(10) Pub. No.: US 2007/0073342 A1
(43) Pub. Date: Mar. 29, 2007

(54) ANNULAR ACCESS DEVICE USING T-ANCHORS

(75) Inventors: Corbett W. Stone, San Diego, CA (US); Jennifer Diederich, Cumberland, RI (US); Bryan D. Knodel, Flagstaff, AZ (US)

Correspondence Address: CARR LLP (IST), 670 FOUNDERS SQUARE, 900 JACKSON STREET, DALLAS, TX, 75202, US

(73) Assignee: Innovative Spinal Technologies

(21) Appl. No.: 11/535,383

(22) Filed: Sep. 26, 2006

Related U.S. Application Data
(60) Provisional application No. 60/720,848, filed on Sep. 27, 2005.
(60) Provisional application No. 60/780,897, filed on Mar. 9, 2006.

(51) Int. Cl. A61B 17/04

(52) U.S. Cl. 606/232

(57) ABSTRACT

Suture locking devices and methods. In one embodiment, the device includes a suture lock, a rotatable actuator adjacent to the lock, and a release mechanism. The lock defines a pair of suture holes which receive sutures attached to tissue (e.g. a spinal disc) of the surgical site. When the actuator rotates the sutures are wound together (preferably around a hub on the lock) thereby securing the plate to the tissue. Furthermore, the release mechanism is coupled to the lock in such a manner that when the release mechanism is actuated the device releases the lock. The release mechanism may include a push button that is biased away from the lock. Also, the actuator may include a thumbwheel and a gear. Further, the gear may also define suture holes. Additionally, a pull ring connected to a pair of loops for drawing the sutures through the instrument may be included.

### CROSS REFERENCE TO RELATED APPLICATIONS

This application relates to co-pending U.S. patent application entitled SYSTEM AND METHOD TO DELIVER ANCHORS INTO ANNULUS TISSUE No. 60/720,848 filed On Sep. 27, 2005, to co-pending U.S. Patent Application entitled ANNULAR ACCESS DEVICE USING T-ANCHORS” No. 60/780,897 filed on Mar. 9, 2006, and to U.S. patent application entitled “ANNULAR ACCESS DEVICES” Ser. No. 11/462,319 filed on Aug. 3, 2006 all of which are incorporated herein as if set forth in full.

### TECHNICAL FIELD

The invention relates generally to medical devices for treatment of spinal injuries and, more particularly, to devices for closing a breach in annulus tissue and/or nucleus tissue of an intervertebral disc.

### BACKGROUND

The spinal column comprises a number of bony vertebrae. Each vertebral body is composed of hard cortical bone on the outside, and less dense cancellous bone on the inside. The top and bottom of the vertebral body are called the endplates. In a healthy state the vertebrae are separated from each other by intervertebral discs, which lie between the respective endplates.

The intervertebral discs are complex structures that support the weight of the body and, with the facet joints, permit a significant range of motion. Each disc is made up of fibrocartilage and has two parts: the nucleus pulposus (the “nucleus”) and the annulus fibrosis (the “annulus”). The nucleus is a gel-like material located in the center of the disc. It has a high water content, which allows it to act as a cushion and distribute loads onto the vertebral body endplates and to the annulus. The annulus is the outer portion of the disc. The annulus consists of 15 to 25 layers of collagen, much like the layers of a truck tire. The structural design allows the annulus to contain the nucleus under pressure, and to help hold the vertebral bodies in place. The annulus also binds the adjacent vertebrae together using collagen fibers that are attached to the vertebrae and cross each other so that half of the individual fibers will tighten as the vertebrae are rotated in either direction, thus resisting twisting or torsional motion.

As the aging process continues, the center of the disc may start to lose water content, making the disc less effective as a cushion. This may cause a displacement of the disc's center (called a herniated or ruptured disc) through a crack in the outer layer. The result is that the disc height is reduced leading to compression of the nerve bundles, causing pain and in some cases damage to the nerves.

Currently, there are many systems and methods at the disposal of a physician for reducing, or eliminating, the pain by minimizing the stress on the nerve bundles. In some instances, the existing disc is removed and an artificial disc is substituted therefore. In other instances, two or more vertebrae are fused together to prevent relative movement between the fused discs.

The ruptured disc should be surgically repaired as quickly as possible and without doing more damage to the surrounding tissue and muscle of the patient unless absolutely necessary. With traditional surgical techniques, relatively large amounts of muscle and tissue must be removed to access the annulus. Such procedures expose the patients to more pain, additional recovery time, and a greater likelihood of infection.

What is needed, therefore, are devices and methods which allow for rapid and secure closure of the disc in a minimally invasive or percutaneous manner.

### SUMMARY

In response to these and other problems, in one embodiment, there is disclosed a suture locking device which includes a suture lock, a rotatable actuator adjacent to the lock, and a release mechanism. The lock defines a pair of suture holes which receive sutures attached to tissue of the surgical site. When the actuator rotates the sutures are wound together thereby securing the plate to the tissue. Furthermore, the release mechanism is coupled to the lock in such a manner that when the release mechanism is actuated the device releases the lock.

These and other features, and advantages, will be more clearly understood from the following detailed description taken in conjunction with the accompanying drawings. It is important to note the drawings are not intended to represent the only aspect of the invention.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods, and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the invention is intended to encompass within its scope such processes, machines, manufacture, compositions of matter, means, methods, or steps.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 is a top view of an embodiment of an anchor delivery device;

FIGS. 2A-B are longitudinal section views of one embodiment of a deployment end of the anchor delivery device;

FIG. 2C is a needle and a T-anchor which has been deployed from an exit port as a result of the actuating device;

FIGS. 3A-C are detail sectional views of one embodiment of an actuating mechanism of the anchor delivery device;

FIG. 4 is one embodiment of a suture locking device;

FIG. 5A is a detailed section view of an actuating mechanism of the suture locking device of FIG. 4;

FIG. 5B is a section view illustrating one embodiment of a distal end of a shaft mechanism coupled to a suture cap of the suture locking device;

FIG. 5C is a transverse cross-section view of the actuating mechanism of the suture locking device;

FIG. 5D is another detailed section view of an actuating mechanism of the suture locking device of FIG. 4;

FIG. 5E is a section view illustrating one embodiment of a distal end of a shaft mechanism releasing the suture cap of the suture locking device;

FIG. 6A is an isometric detail view of one embodiment of a suture cap;

FIG. 6B is section view of the suture cap;

FIG. 6C is a perspective view of the distal end of the distal end of the shaft mechanism with a suture cap lid transparent;

FIGS. 7A-C are exploded views of the suture cap;

FIG. 8 illustrates a method for using certain aspects of the present invention;

FIGS. 9A-E illustrate one method of preparing a surgical site and placement of one embodiment of the present invention;

FIG. 10A is a detailed view showing a distal end of the anchor delivery device placed next to an exposed exterior surface of the annulus;

FIG. 10B is a detailed view showing where a T-anchor has been deployed into the nucleus and is protruding beyond an outer tube;

FIG. 10C is a detailed view showing where both T-anchors have been deployed into the nucleus;

FIG. 10D is a detailed view showing where both T-anchors have been deployed and the anchor delivery device is being removed;

FIG. 10E is a detailed view showing where a suture lock device is about to be deployed;

FIG. 10F is a detailed view showing where the sutures are pulled up through the suture lock device;

FIG. 10G is a detailed view showing a suture cap being released from the suture lock device and the sutures cut;

FIG. 11A is a detailed view showing a distal end of the anchor delivery device placed next to an exposed exterior surface of the annulus;

FIG. 11B is a detailed view showing where a T-anchor has been deployed into the annulus and is protruding beyond an outer tube;

FIG. 11C is a detailed view showing where both T-anchors have been deployed into the annulus;

FIG. 11D is a detailed view showing where both T-anchors have been deployed and the anchor delivery device is being removed;

FIG. 11E is a detailed view showing where a suture lock device is about to be deployed;

FIG. 11F is a detailed view showing where the sutures are pulled up through the suture lock device;

FIG. 11G is a detailed view showing a suture cap being released from the suture lock device and the sutures cut;

FIGS. 12A-D are detailed views of one embodiment of a T-anchor;

FIGS. 13A-D are detailed views of an alternative embodiment of a T-anchor;

FIGS. 14A-D are detailed views of an alternative embodiment of a T-anchor; and

FIGS. 15A-D are detailed views of an alternative embodiment of a T-anchor.

### DETAILED DESCRIPTION

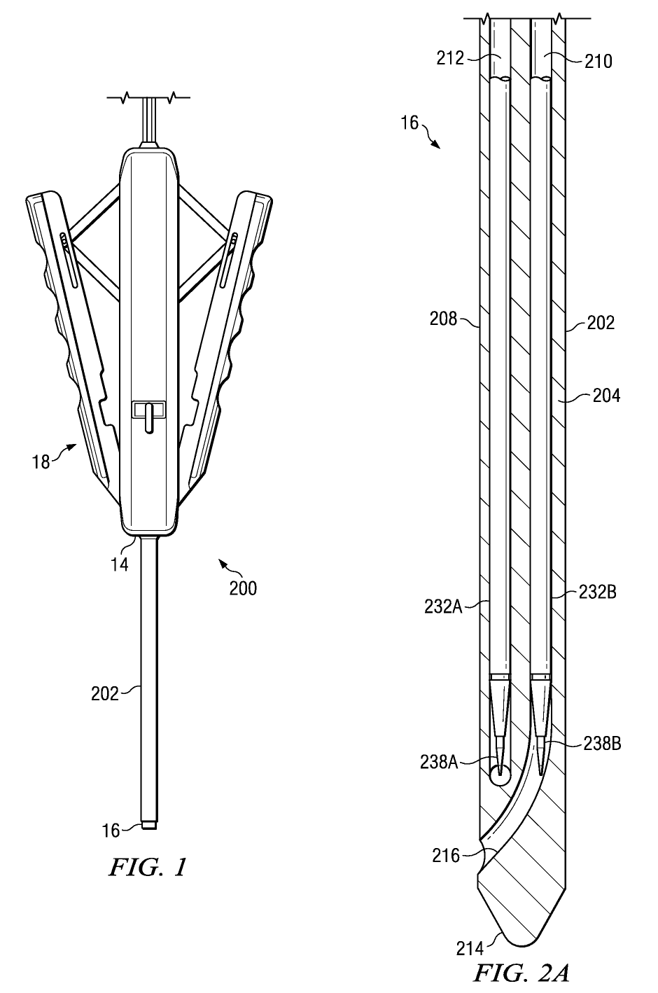

FIG. 1 depicts a top view of one embodiment of an anchor delivery device 200. The anchor delivery device 200 can comprise an outer tube or cannula 202, having a proximal end 14 and a distal end or deployment end 16. In some embodiments, an actuating mechanism 18 may be coupled to the proximal end of the cannula. In certain embodiments, the outer cannula 202 may also contain one or more needle delivery lumens (not shown in FIG. 1) running longitudinally from the actuating mechanism 18 to the deployment end 16.

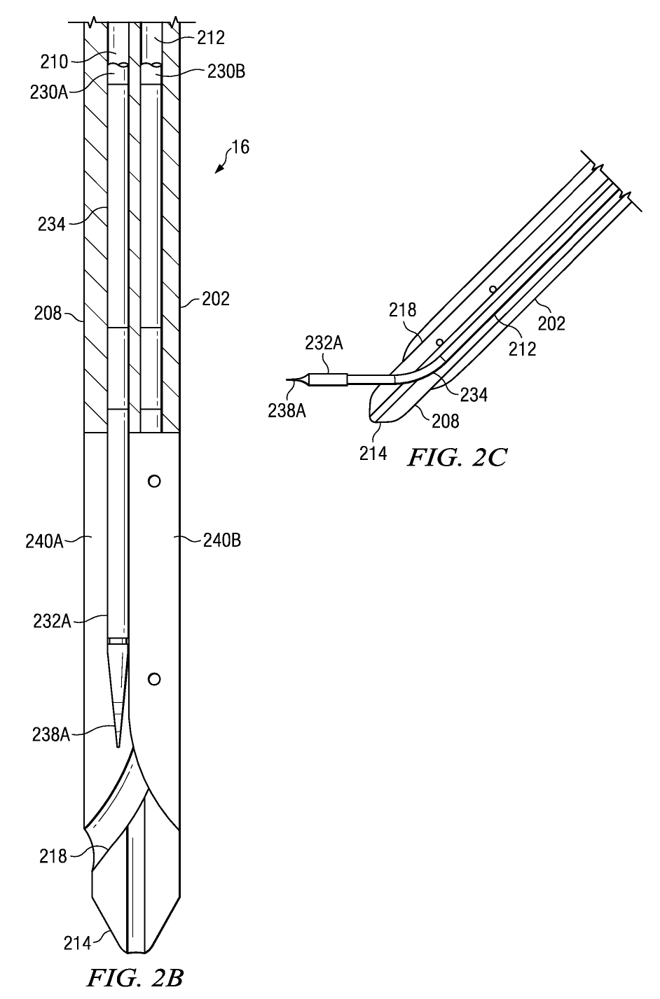

Turning now to FIGS. 2A and 2B, there are longitudinal section views of one embodiment of the deployment end 16. FIG. 2A illustrates a section cut in one direction and FIG. 2B illustrates a section cut in another direction. In some embodiments, the outer tube 202 of the anchor delivery device 200 can comprise a central channel 204 running lengthwise therethrough, and an inner assembly disposed within the central channel 204. In certain embodiments, the inner assembly can comprise an inner tube 208 having at least two internal lumens 210, 212 running lengthwise therethrough. In some embodiments, the inner tube 208 may extend beyond the distal end of outer tube 202. A tip 214 may be formed at a distal end of inner tube 208. In some embodiments, the tip 214 may be generally conical having a rounded point so that inner tube 208 may function as a blunt obturator. In other words, the pointed tip allows the obturator to puncture tissue, such as the annulus. In other embodiments, there may be only an outer tube 202 without using the inner tube 208. In yet other embodiments, the tip 214 may not be rounded and would not be used as an obturator.

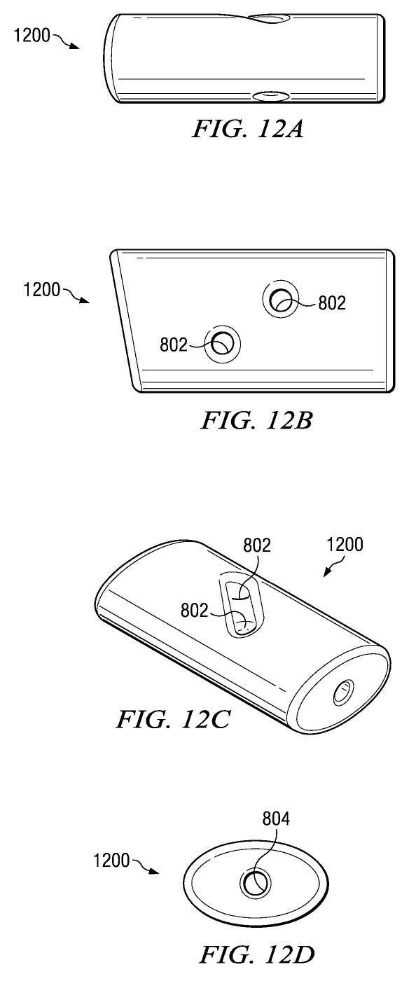

In certain embodiments, the internal lumens 210, 212 can run longitudinally, but then curve to connect to exit ports 216, 218, respectively formed in sides the inner tube 208 behind the tip 214. In certain embodiments, there may be anchors 232a and 232b positioned within the internal lumens 212 and 210, respectively. In certain embodiments, these anchors 232a-232b may be T-anchors. T-anchors are further described with reference to FIGS. 12-15. In some embodiments, the anchors 232a-232b may be hollow and/or flexible. In certain embodiments, the anchors 232a-232b may be fabricated from a flexible polymer or other bio-resorbable plastic material. In certain embodiments, flexible drive rods 234 may be slidably disposed within the internal lumens 212, 210 between the anchors 232a-232b and pusher tubes 230a and 230b. In certain embodiments, the drive rods 234 may be fabricated from a flexible metal alloy such as Nitinol, a nickel-titanium alloy.

In some embodiments, sutures (not shown) may be attached to each anchor 232a, 232b and may run through the drive rods 234 and pusher tubes 230a-230b. In some embodiments, there may be needles 238a and 238b which pass through the anchors 232a-232b. In certain embodiments, the needles 238a and 238b may extend from the forward tip of the anchors to assist in penetration of the annulus and insertion of the anchors in the annulus. In some embodiments, there may be filler plates 240a and 240b which separate the channels 210, 212 as the channels cross over each other to connect to exit ports 216 and 218.

In certain embodiments, there may be an actuating device 18 (FIG. 1) which longitudinally moves the pusher tubes 230a-230b within the internal lumens 210 and 212, respectively. As the pusher tubes 230a-230b move, they move the drive rods 234, which, in turn, drive the anchors 232a-232b out of the respective ports 218 and 216, respectively. Accordingly, when the anchors are driven out of their respective ports, they may be deployed into the nucleus or the annulus.

Referring now to FIG. 2C, there is illustrated a needle 238a and a T-anchor 232a which has been deployed or pushed out of the exit port 218 as a result of the actuating mechanism actuating or pushing on the pusher tube 230b (not shown). Note that the drive rod 234 is flexible so that it bends as the curved portion of the internal lumen 212.

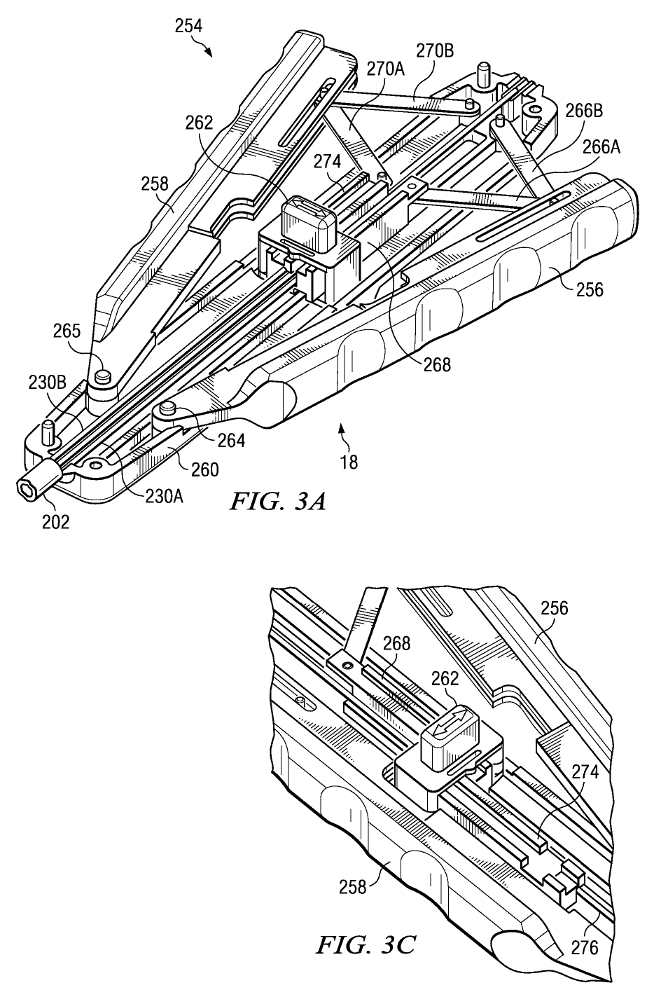

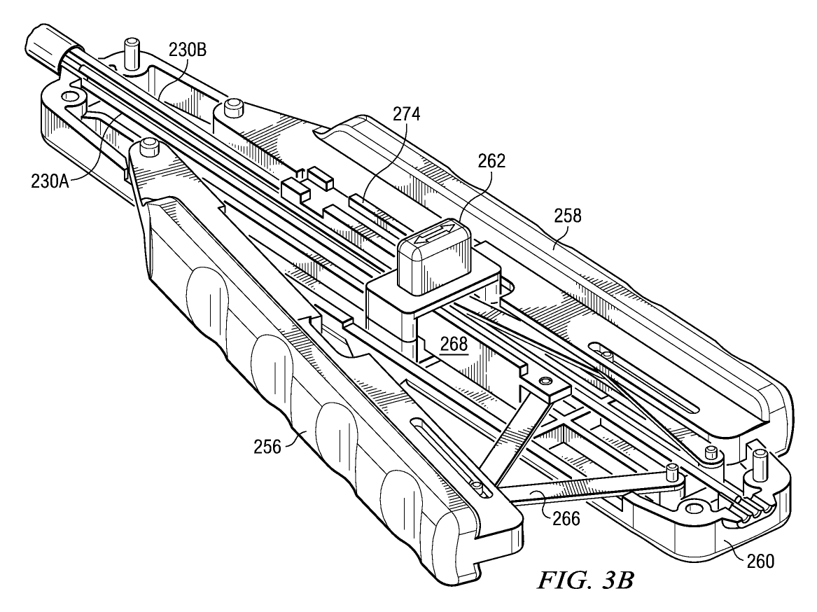

Turning now to FIG. 3A, there is one embodiment of an actuating device 18. In this illustrative figure, one half of a handle body 260 is removed for clarity. In certain embodiments, an actuation handle or lever 256 may be pivotally coupled to the handle body 260 at a pivot pin 264. In some embodiments, link members 266a and 266b may operatively couple actuation lever 256 to a shuttle 268. The shuttle 268 may be slidably mounted within the handle body 260. In certain embodiments, the shuttle 268 may engage a pusher tube 230a that extends into the outer cannula 202 to drive one of the anchors as discussed above.

In a similar fashion, an actuation lever 258 may be pivotally mounted on the handle body 260, and link members 270a-270b may operatively couple the actuation lever 258 to a shuttle 274 which may be slidably mounted within the handle body 260 as illustrated. In certain embodiments, the shuttle 274 may engage a pusher tube 230b that extends into the outer cannula 202 to drive another one of the anchors, as discussed above.

In certain embodiments, there may be a selector switch 262, which may be operated from either side of handle assembly 254. The selector switch allows a surgeon to selectively engage one or both shuttles 268, 274. Consequently, allowing the anchor delivery device 200 to drive one or both of the anchors depending on the position of the selector switch.

For instance, FIG. 3B illustrates a situation where only the actuation lever 258 is pressed into the handle assembly 254. Thus, the linkage comprising links 270a and 270b (not shown) can be extended and drive the shuttle 274 forward. This action can move the pusher tube 230b, which in turn, deploys an anchor (not shown) at the deployment end of the device. Handle 256 remains extended from the handle body 260, link members 266a-266b remain un-extended, and shuttle 268 remains in its original, rearward position. FIG. 3C illustrates in detail one embodiment of the action of the selector switch 262 engaging the shuttle 274, but leaving behind shuttle 268.

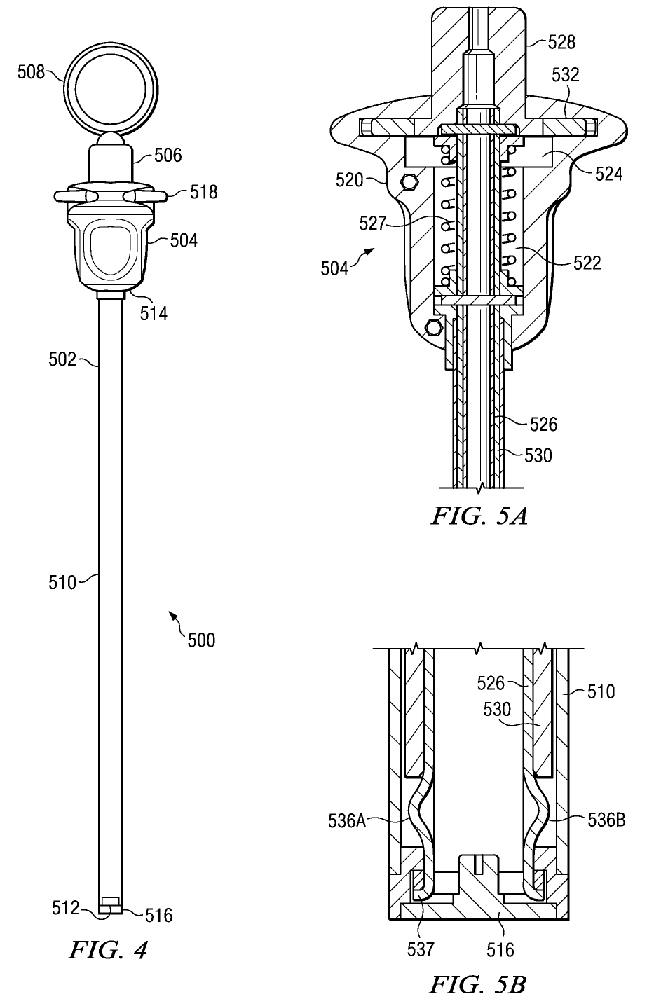

Referring now to FIG. 4, there is illustrated one embodiment of a suture locking device 500. In this exemplary embodiment, the suture locking device 500 may comprise a shaft mechanism 502, a suture actuating mechanism 504, a release mechanism 506, and a pull ring 508. The shaft mechanism 502 may comprise an outer tube 510 and an inner tube (not shown) having a distal end 512 and a proximal end 514. In this embodiment, the shaft mechanism 502 can deliver a suture lock or suture cap 516 which may be releasably coupled to the distal end 512. In some embodiments, the suture cap could be fabricated from a flexible polymer or other bio-resorbable plastic material.

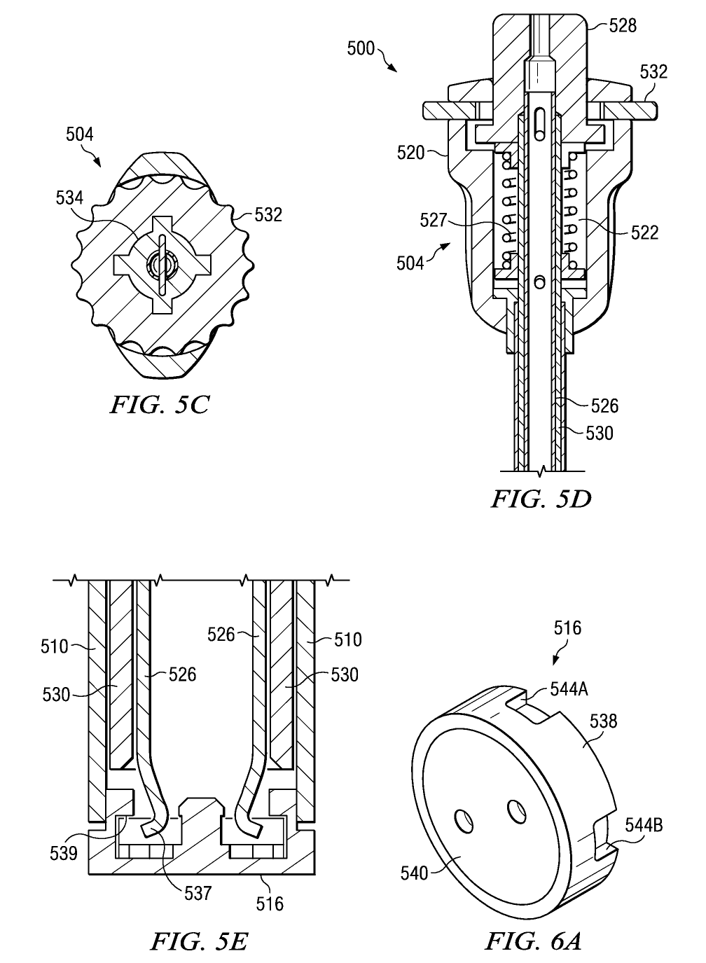

FIG. 5A is a detailed section view of the actuating mechanism 504. In this embodiment, there is a housing 520 which may also serve as a handle. The housing 520 can have a central bore 522, which widens at its proximal end to form a shoulder 524. An inner or center tube 526 can run longitudinally from the distal end of the shaft mechanism 502 to a cap release button 528. The cap release button 528 may slidingly engage the center tube 526. In certain embodiments, the cap release button comprises a circular top portion and a lower plate portion. The cap release button may have a central bore for coupling with the suture pull ring 508 (FIG. 4). In certain embodiments, the cap release button may also be coupled to a release tube 530, which is disposed concentrically between the center tube 526 and the outer tube 510.

In certain embodiments, a helical spring 527 disposed within the central bore 522 biases the cap release button 528 towards the proximal direction. Rotatably mounted within the suture actuating mechanism 504 can be a thumb wheel 532 which may be coupled to release tube 530 via the cap release button 528. Rotation of the thumb wheel 532 causes concomitant rotation of the center tube 526 and a portion of the suture cap 516 which may be coupled to the distal end of the center tube 526.

FIG. 5B is a section view illustrating one embodiment of a distal end of the shaft mechanism coupled to the suture cap 516. In this embodiment, there may be a center or inner tube 526, the release tube 530, and the outer tube 510. In one embodiment, the inner tube 526 may be coupled to a plurality of bent coupling arms (arms 536a and 536b are shown). The coupling arms may be bent so that they extend into the path of the release tube 530. At their distal ends, the coupling arms may also be bent to form a hook 537 which couples to the suture cap 516. In yet another embodiment, the inner tube may have longitudinal slits extend up from the distal end to provide additional flexibility to the coupling arms 536. The inner tube may then be bent in a manner similar to the coupling arms. Thus, as illustrated, the coupling arms (or, alternatively, the inner tube) may engage the suture cap 516.

Turning now to FIG. 5C, there is a transverse cross-section view of the suture actuating mechanism 504. In FIG. 5C, the thumb wheel 532 is illustrated in a plan view. Also illustrated is a bottom plate 534 of the cap release button 528. In this illustrated embodiment, the bottom plate 534 has four keys evenly spaced around the periphery of the plate. The four keys mate with corresponding slots of the thumb wheel 532. Thus, as can be seen from FIGS. 5A and 5C, when the cap release button 528 is biased in a first or proximal position, the keys of the bottom plate may engage the slots of the thumb wheel 532. When the keys and slots are engaged, turning the thumb wheel turns the cap release button, which subsequently turns the center tube 526. In contrast, when the cap release button 528 is in a second or distal position (in which the biasing force of the spring 527 has been overcome), the keys of the bottom plate are disengaged from the slots of the thumb wheel 532. Accordingly, with the cap release button depressed, turning the thumb wheel 532 results in no rotation of the cap release button 528 or the center tube 526.

Pressing on the release button 528 may move the release tube 530 in a distal direction. When the release tube moves 530 down towards the distal end of the shaft mechanism, the release tube moves the coupling arms 536 in an inward direction. The hooks 537 may also be moved in an inward direction, which then releases the suture cap 516 from the shaft mechanism as illustrated in FIGS. 5D-E.

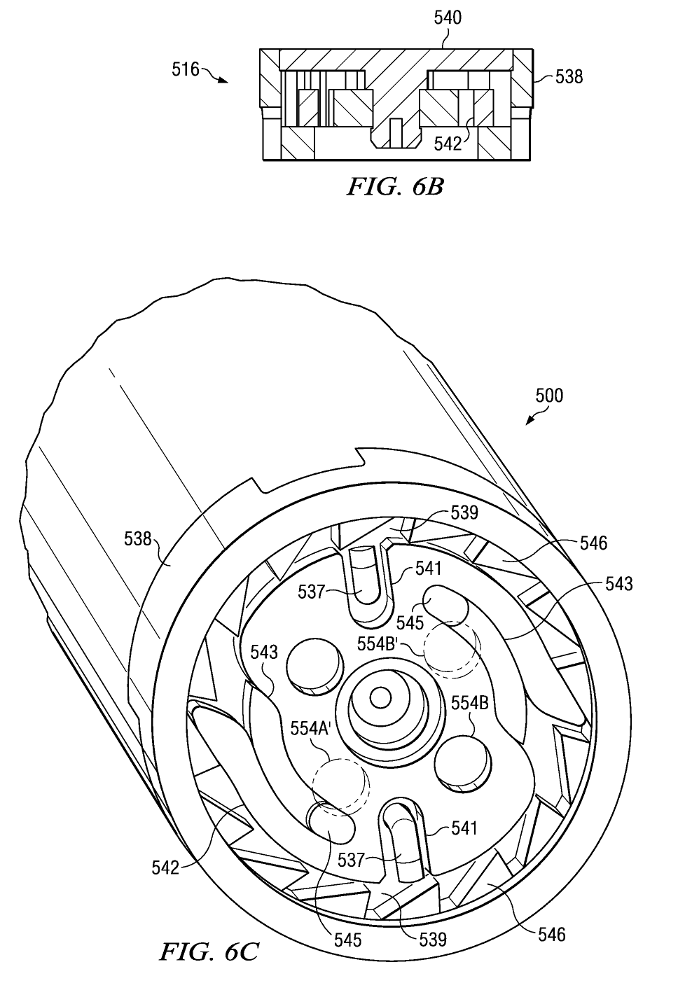

FIG. 6A is an isometric detail view of one embodiment of a suture cap 516. On the other hand, FIG. 6B is a section view of the suture cap 516. Turning now to both FIGS. 6A and 6B, it can be seen that in this embodiment, the suture cap 516 can comprise a cap housing 538, a cap lid 540 and a gear 542. In some embodiments, the cap housing 538 can have four holding slots 544a-544d (554a and 554b are illustrated) evenly spaced around the periphery thereof. The holding slots 544a-544b may mate with tabs formed on the distal end of the outer tube 510 for rotationally stabilizing the suture cap with the outer tube 510.

FIG. 6C illustrates the distal end of the instrument 500 with the suture cap 516 transparent and the gear 542 visible. More particularly, FIG. 6C shows the hooks 537 engaging a land 539 on the inside of the cap housing 538 which allows the hooks 537 to retain the cap until the hooks 537 are moved in by release tube 530. Moreover, FIG. 6C shows that the gear 542 includes an alignment slot 541 for the hooks 537 so that during assembly of the instrument 500, the gear 542 can be aligned with the hooks 537 and assembled into the instrument. Furthermore, the gear 542 includes slots 543 which partially define the teeth of the gear and which allow drive pins 545 of center tube 526 to engage and drive the gear 542. More specifically, the elongate slots 543 may provide some flexibility to the gear teeth to aid in their engagement of the cap teeth 546 to allow rotation in one direction but not the other. Thus, when the center tube 526 turns via thumbwheel 532 (FIG. 5D), the drive pins 545 cause the gear 542 to turn. FIG. 6C also shows that the gear 542 may rotate from one position where the suture holes 554A and 554B may initially be aligned with the suture holes 550A and 550B of the suture cap 540 to a second position where the gear suture holes 554A and 554B are represented as references 554A′ and 554B′.

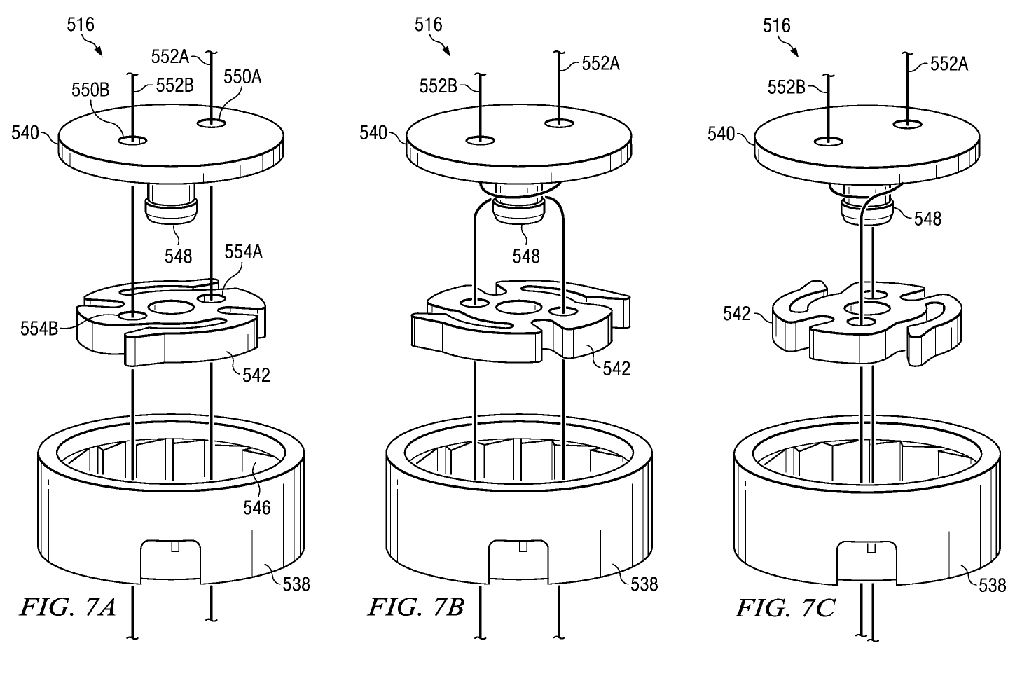

Turning now to FIG. 7A, there is an exploded view of the suture cap 516. As illustrated in this view, there can be a plurality of teeth 546 on the inside surface of the cap housing 538. The teeth 546 engage the gear 542 in such a manner as to allow rotation in one direction, but not the other. As illustrated, the gear 542 may be rotatably coupled to the cap lid 540 via a center hub 548 protruding from the cap lid. In this embodiment, the cap lid 540 may have two suture holes 550a and 550b for passing two ends of sutures 552a and 552b therethrough. In certain embodiments, the suture cap 516 can include a gusset around the end of the hub 548 which can assist in retaining the sutures on the hub 548 during, and after, the sutures are wound around the hub 548. Further, in some embodiments, the gear 542 can also have a set of suture holes 554a and 554b for passing the two ends of the sutures 552a and 552b.

As will be explained below in greater detail, the distal end of device 500 may be positioned adjacent the site of a surgical procedure, such as an annulotomy. At the completion of a surgical procedure, the surgeon can rotate the thumbwheel knob 532 (FIG. 5A), thereby rotating the inner tube 526 (FIG. 5A) which may be coupled to the gear 542. Thus, the sutures 552a and 552b passing through suture holes 550a and 550b of the cap lid 540 and suture holes 554a and 554b of the gear 542 may be wound together over or around the hub 548 as illustrated in FIG. 7B.

FIG. 7B is an exploded view of the suture cap 516, where the gear 542 is rotated 45 degrees with respect to the cap lid 540. Similarly, FIG. 7C is an exploded view of the suture cap 516, where the gear 542 is rotated 90 degrees with respect to the cap lid 540 which illustrates more suture winding around the center hub 548. Once the winding is complete, the surgeon can the press the release button 528 (FIG. 5A) to extend the release tube 530 in a distal direction and release the suture cap 516 from the shaft mechanism. The device 500 may then be removed from the surgical site and the sutures cut.

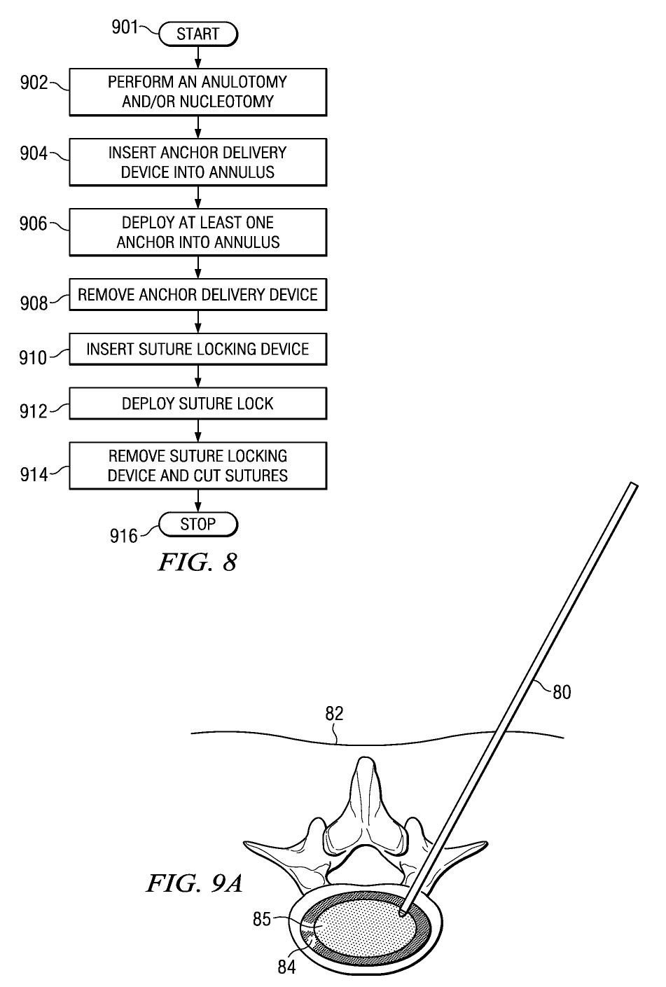

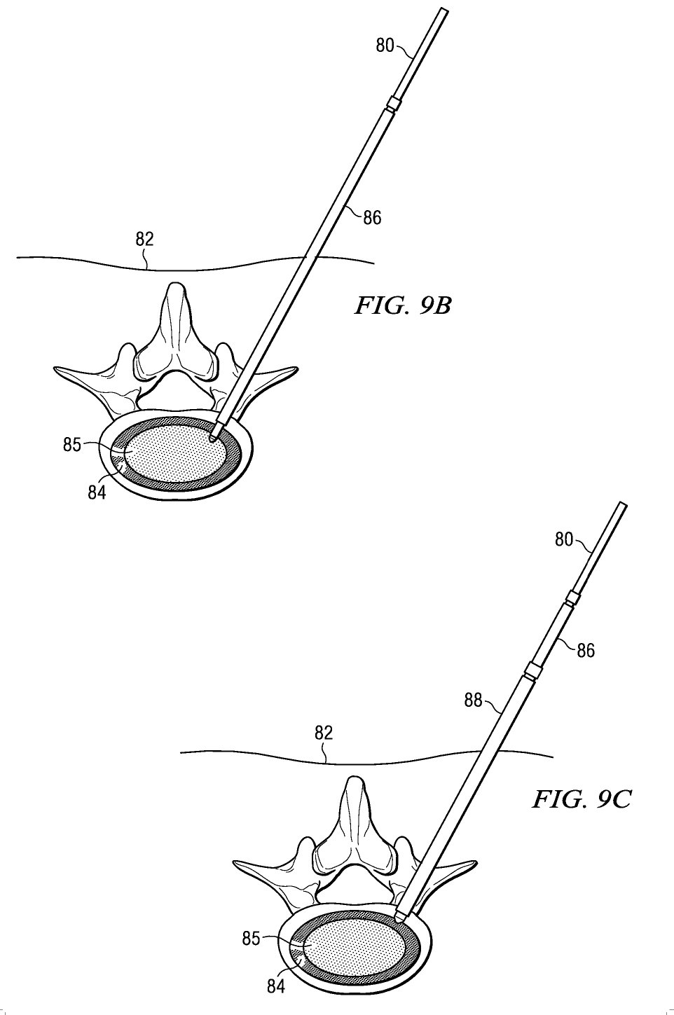

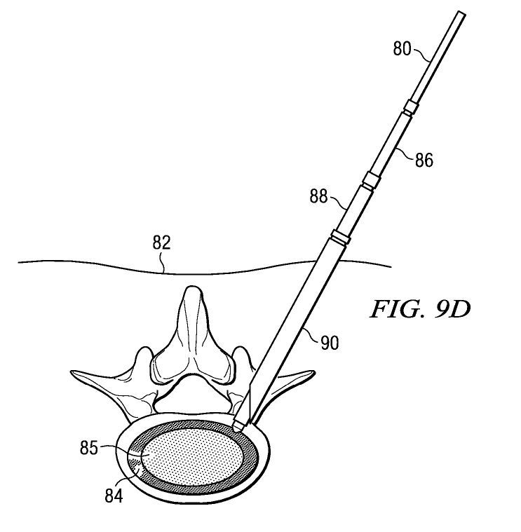

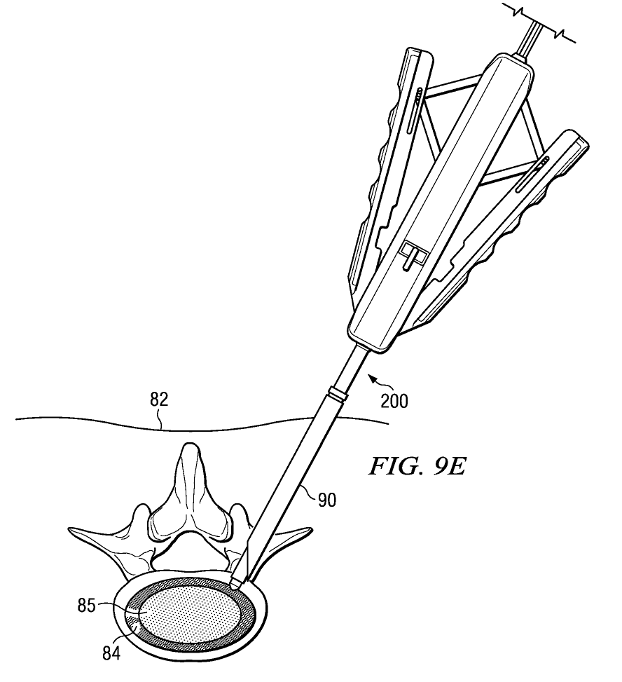

Referring now to FIGS. 8-9, one manner of using the anchor delivery device 200 will now be described. FIG. 8 illustrates a general method which could employ one or more aspects of the present invention. FIGS. 9A through 9E illustrate one method of preparing a surgical site and placement of one embodiment of the present invention.

Turning now to FIG. 8, there is illustrated a method for using certain aspects of the present invention. Details regarding the method will be explained below. The procedure begins at step 901 and flows to step 902. In step 902, an annulotomy may be performed. In step 904, the anchor delivery device may then deploy the anchors into the annulus in step 906. After deployment of the anchors, in step 908, the anchor delivery device may be removed. In step 910, a suture locking device may be inserted adjacent to the annulus, where the suture cap is deployed (step 912). The suture locking device may then be removed and the sutures may be cut (step 914). The procedure stops at step 916 and closure may be accomplished in a conventional manner.

One aspect of performing an annulotomy may be illustrated in FIGS. 9A-9E. As illustrated in FIG. 9A, a k-wire 80 may be advanced percutaneously in a lateral or posterolateral approach. The surgeon may use fluoroscopic techniques to guide him through the muscles and tissues 82 to the annulus 84. The surgeon may drive the k-wire through the annulus 84 and into the nucleus disc space 85. An obturator 86 may then be advanced over the guide wire to the outer surface of the annulus as illustrated in FIG. 9B. A first dilator 88 having a larger radius than the obturator 86 may then be advanced over the obturator as illustrated in FIG. 9C. The first dilator 88 may be either straight or beveled as illustrated. In certain embodiments, a plurality of dilators may be used to provide a progressive larger access to the surgical site. As illustrated in FIG. 9D, a second dilator 90, having a larger radius than the first dilator may be advanced over the first dilator 88. The second dilator may also be straight or beveled. The k-wire 80, the obturator 86, and the first dilator 88 may then be removed leaving the second dilator 90 in place and exposing the surface of the annulus 84.

FIG. 9E illustrates a situation where the k-wire 80, the obturator 86, and the first dilator 88 have been removed and an anchor delivery device 200 has been inserted into the second dilator 90 such that the distal end of the annular access device is adjacent to the surface of the annulus.

In certain embodiments, an incision into the annulus 84 may be performed with a trephine (not shown). In some alternative embodiments, an obturator may be used to perform the annulotomy. Such an incision may be made through the outer surface of the annulus of the disc. In other embodiments, a cannula or dilator may include an extendable or fixed sheath or guard (not shown) to protect the annulus during the surgical procedure. In such embodiments, the cannula may be advanced into the disc space at a depth beyond the surface of the annulus.

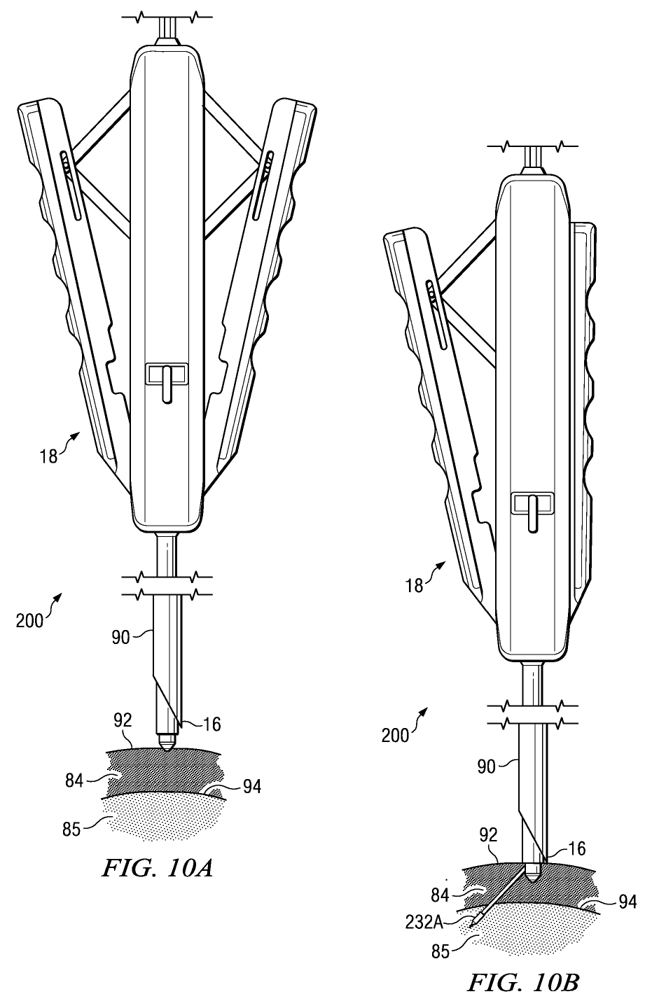

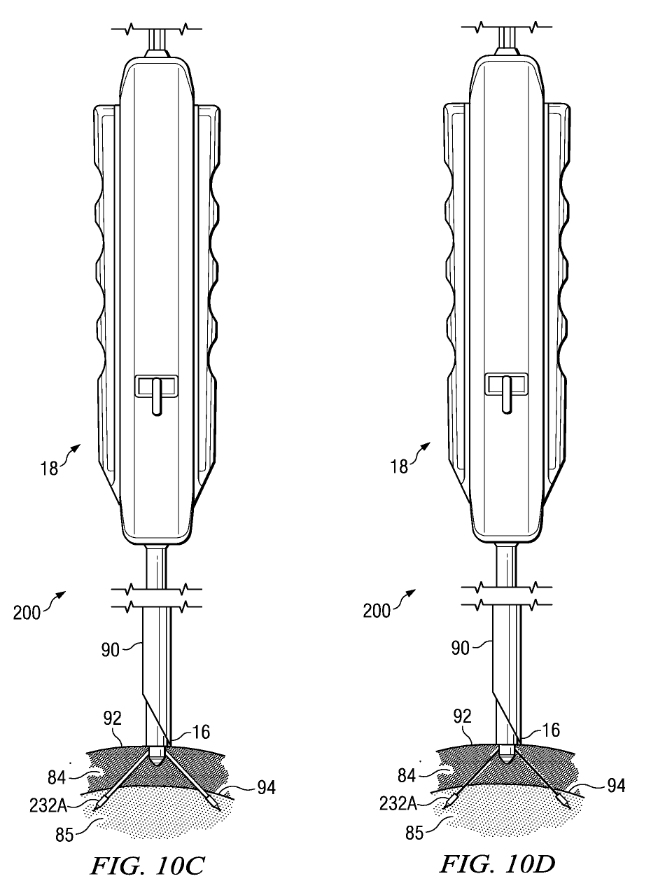

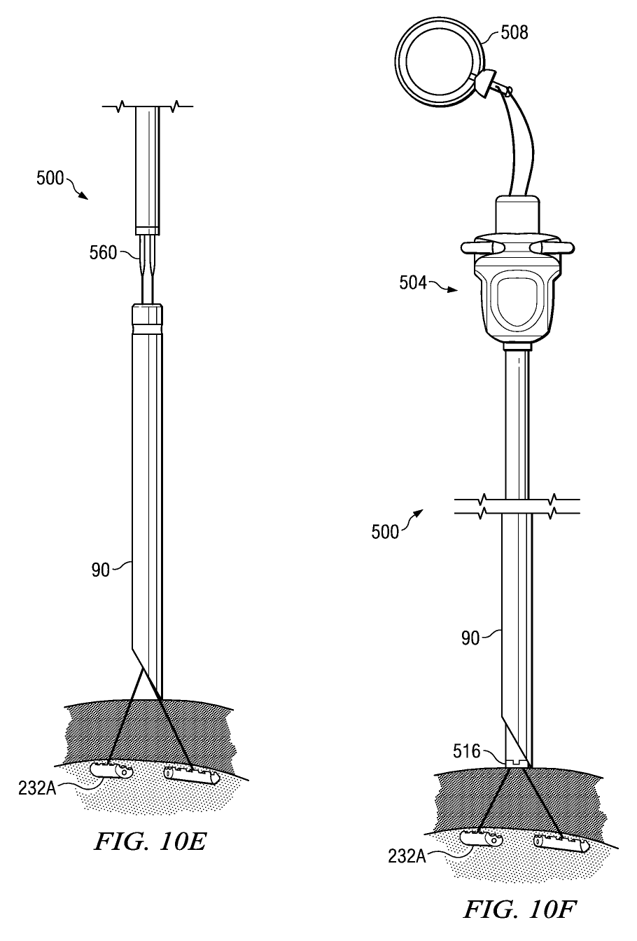

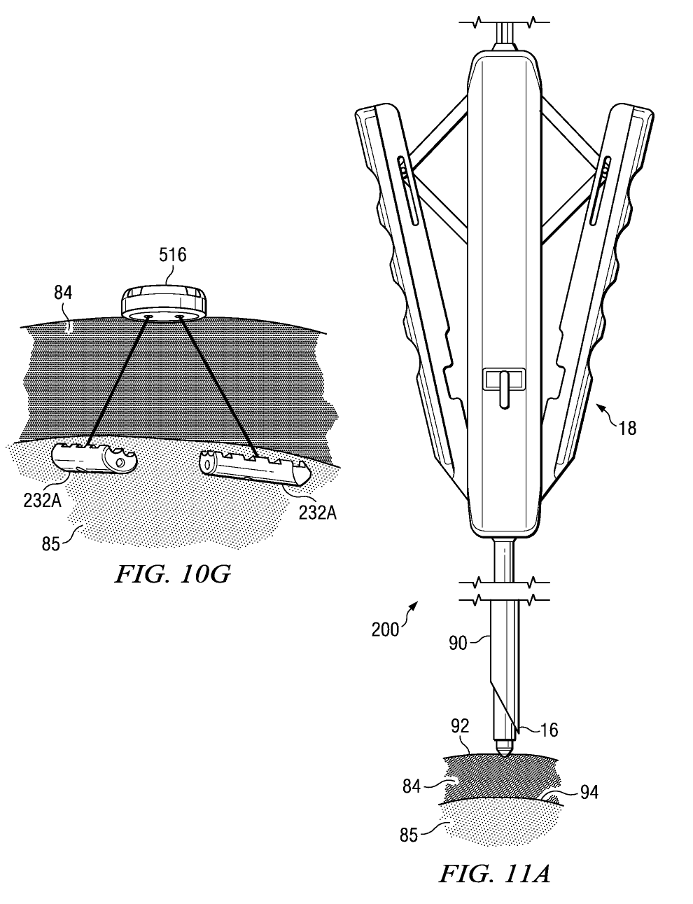

FIG. 10A is a detailed view showing the distal end 16 of the anchor delivery device 200 placed next to an exposed exterior surface 92 of the annulus 84. For illustrative purposes, line 94 represents a demarcation between the annulus 84 and a nucleus 85. However, an actual disc would not have a clear demarcation line between the annulus 84 and the nucleus 85. As previously discussed, in certain embodiments, the anchor delivery device 200 may be placed within the second dilator 90. In this situation, the needles and the anchors (T-anchors) may be within the respective needle lumens in a first or retracted position.

In FIG. 10B, the distal end of the anchor delivery device 200 is shown where a T-anchor 232a has been deployed into the nucleus 85 and is protruding beyond the outer tube. In this figure, one of the actuation levers may have been pressed which has caused one of the T-anchors to deploy in the nucleus 85. The T-anchor can penetrate the annulus 84 to get into the nucleus 85. As previously explained in reference to FIGS. 2A and 2B, when an actuating lever or handle of an actuating device 18 is depressed, a corresponding linkage can move a shuttle longitudinally towards the distal end 16. The shuttle can move a pusher tube within one of the internal lumens. As the pusher tube moves, it also can move a drive rod, which, in turn, drives the T-anchor 232a out of the side port.

FIG. 10C, therefore, illustrates a situation where both actuation levers may have been pressed. Consequently, both T-anchors 232a have been deployed into the nucleus 85.

FIG. 10D illustrates a situation where the T-anchors have been deployed and the anchor delivery device is being removed. Note that the sutures may be attached to the T-anchors as the device is being removed.

In FIG. 10E, the suture lock device is about to be deployed. In this embodiment, one end of a first suture which is coupled to the first T-anchor can be coupled to a loop 560 hanging out of the suture lock device. The second end of the first suture can then be coupled to a second loop 560 hanging out of the suture lock device. Similarly, one end of a second suture which is coupled to the second anchor may be coupled to the first loop 560 hanging out of the suture lock device. The second end of the first suture is then coupled to the second loop 560 hanging out of the suture lock device as illustrated.

After the loops have been coupled to the sutures, the suture lock device 500 may be inserted into the dilator as is illustrated in FIG. 10F. Once the suture lock device 500 has been fully inserted into the dilator, the pull ring 508 (which may be coupled to threads which are attached to the loops), may then be pulled. When the pull ring 508 is pulled the sutures may be pulled up through the suture lock device 500 as illustrated in FIG. 10F. Each suture can be attached to the middle of a T-anchor. This feature enables the t-anchors to securely attach to the inside of the annulus 84 within the nucleus 85.

As previously described, the thumb wheel may then be turned which will wrap the sutures around the suture cap 516. The suture cap 516 may then be released from the suture lock device 500 and the sutures cut as illustrated in FIG. 10G. The T-anchors 232a can be secured to the annulus 84, but can be located in the nucleus 85. Thus, a surgical closure of the annular breach may be provided.

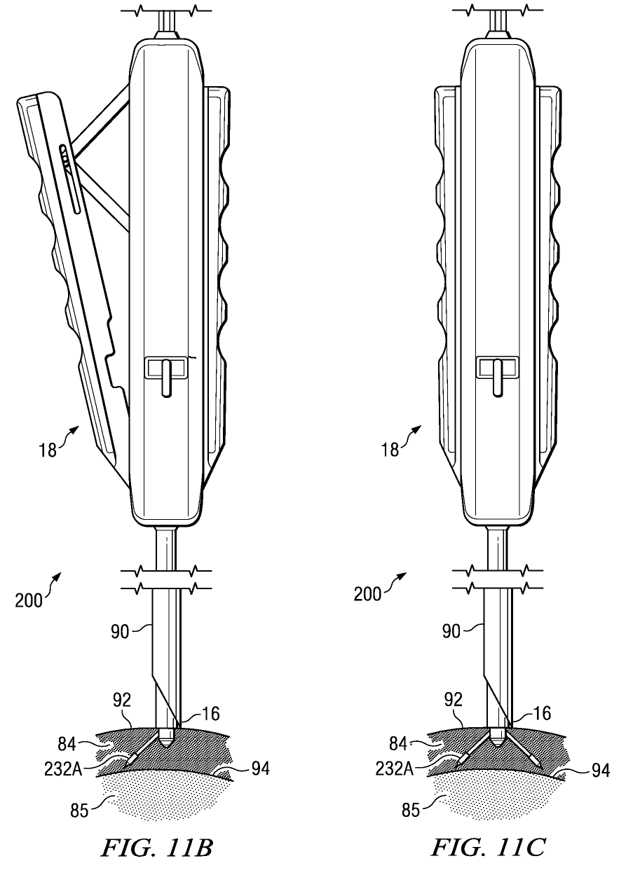

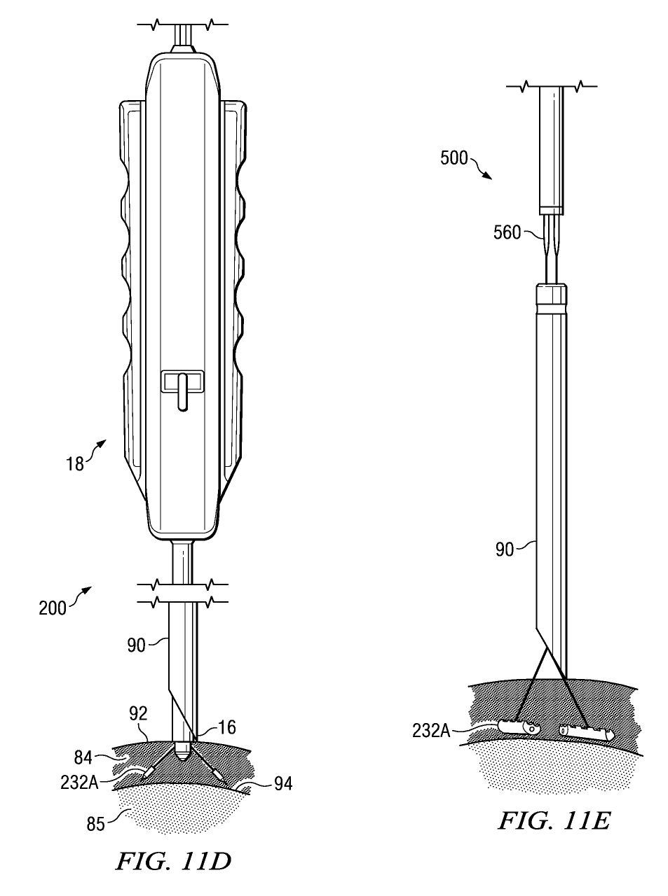

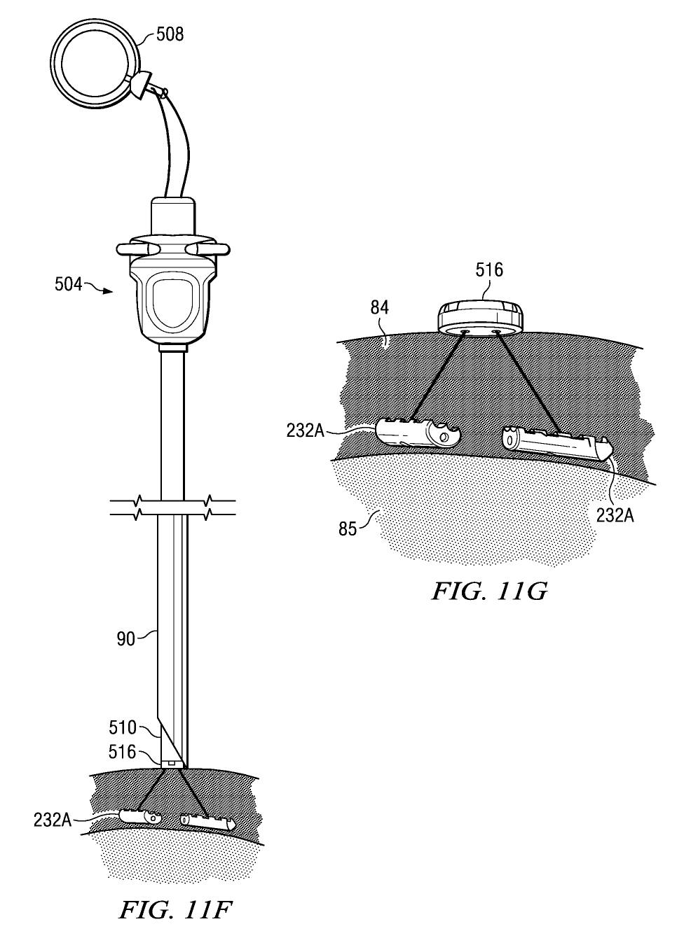

Similarly, FIGS. 11A-G illustrate certain embodiments, wherein the T-anchors 232a are deployed in the annulus 84. FIG. 11A is a detailed view showing a distal end 16 of the anchor delivery device 200 placed next to an exposed exterior surface of the annulus 84. FIG. 11B is a detailed view showing where a T-anchor 232a has been deployed into the annulus 84 and is protruding beyond an outer tube. FIG. 11C is a detailed view showing where both T-anchors 232a have been deployed into the annulus 84. FIG. 11D is a detailed view showing where both T-anchors have been deployed and the anchor delivery device 200 is being removed. FIG. 11E is a detailed view showing where a suture lock device is about to be deployed. FIG. 11F is a detailed view showing where the sutures are pulled up through the suture lock device 500. FIG. 11G is a detailed view showing a suture cap 516 being released from the suture lock device 500 and the sutures cut. Each suture may be attached to the middle of a T-anchor. This feature enables the T-anchors to securely attach to the annulus 15 84. In FIG. 11G, the T-anchors are secured in the annulus 84, and a surgical closure of the annular breach may be provided.

FIGS. 12A-D are detailed views of one embodiment of a T-anchor 1200. FIG. 12A is a side view of the T-anchor 1200. FIG. 12B is a top view of the T-anchor 1200. Two holes 802 on the top of the T-anchor 1200 indicate where the sutures may be secured. FIG. 12C is an isometric view of the T-anchor 1200. FIG. 12D is a front view of the T-anchor 1200. A hole 804 in the front of the T-anchor 1200 indicates where a needle may be inserted into the T-anchor. As previously described, a needle can be used to penetrate the annulus.

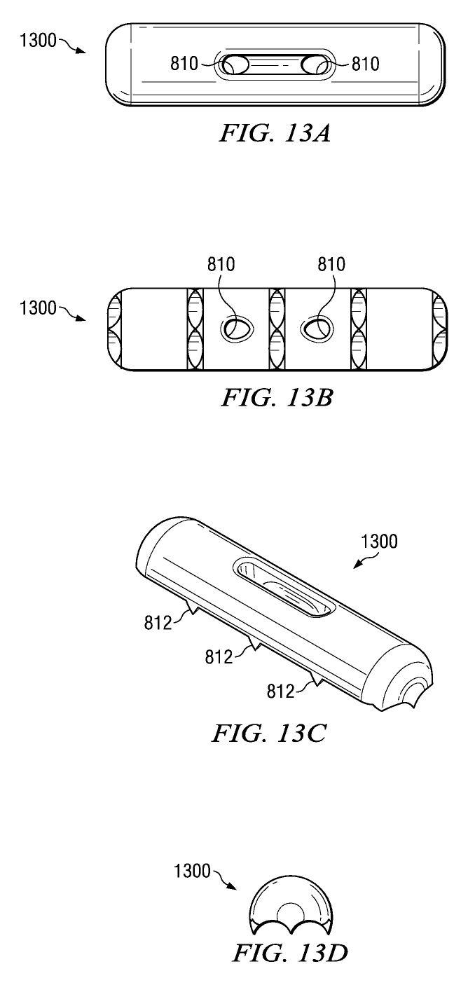

FIGS. 13A-D are detailed views of an alternative embodiment of a T-anchor 1300. FIG. 13A is a top view of the T-anchor 1300. FIG. 13B is a bottom view of the T-anchor 1300. Two holes 810 on the top of the T-anchor 1300 indicate where the sutures may be secured. FIG. 13C is an isometric view of the T-anchor 1300. Fingers 812 on the bottom of the T-anchor 1300 may be used to anchor the T-anchor into the annulus. FIG. 13D is a front view of the T-anchor 1300.

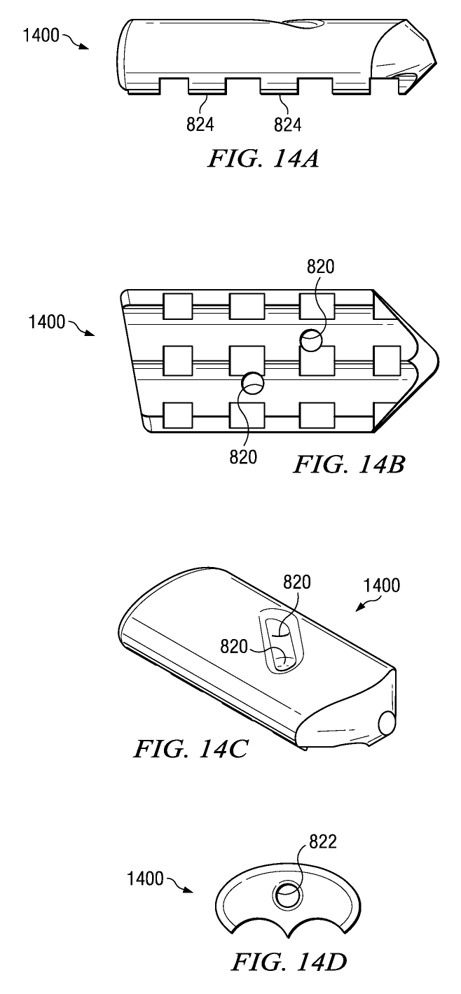

FIGS. 14A-D are detailed views of an alternative embodiment of a T-anchor 1400. FIG. 14A is a side view of the T-anchor 1400. Fingers 824 on the bottom of the T-anchor 1400 may be used to anchor the T-anchor into the annulus. FIG. 14B is a bottom view of the T-anchor 1400. FIG. 14C is an isometric view of the T-anchor 1400. Two holes 820 on the top of the T-anchor 1400 indicate where the sutures may be secured. FIG. 14D is a front view of the T-anchor 1400. A hole 822 in the front of the T-anchor 1400 indicates where a needle can be inserted into the T-anchor.

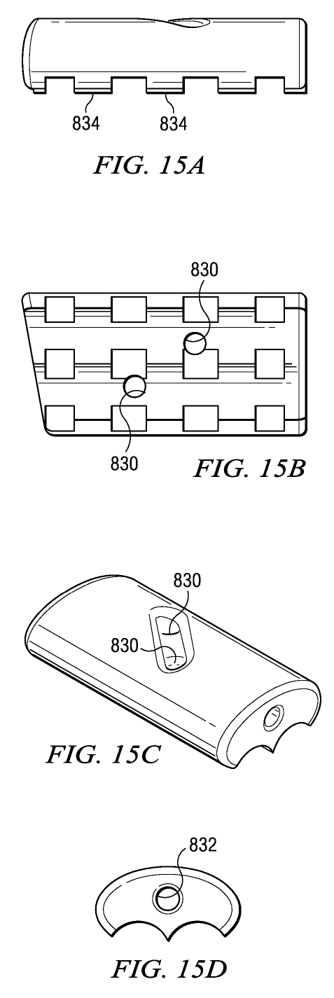

FIGS. 15A-D are detailed views of an alternative embodiment of a T-anchor 1500. FIG. 15A is a side view of the T-anchor 1500. Fingers 834 on the bottom of the T-anchor 1500 may be used to anchor the T-anchor into the annulus. FIG. 15B is a bottom view of the T-anchor 1500. FIG. 15C is an isometric view of the T-anchor 1500. Two holes 830 on the top of the T-anchor 1500 indicate where the sutures may be secured. FIG. 15D is a front view of the T-anchor 1500. A hole 832 in the front of the T-anchor 1500 indicates where a needle can be inserted into the T-anchor.

Other embodiments may include:

1. An apparatus for performing a surgical operation on an intervertebral disc wherein the disc has an annulus on which an annulotomy may be performed, the apparatus comprising a cannula having a proximal end and a distal end, an obturator coupled to the distal end of the cannula and being adapted for insertion into the annulotomy, an annulus engaging member operatively coupled to the obturator and adapted to engage the annulus, and an actuator operatively coupled to the member in such a manner that when the actuator is actuated the member engages the annulus thereby securing the apparatus to the annulus.

2. The apparatus of Embodiment 1 wherein the annulus engaging member comprises an anchor.

3. The apparatus of Embodiment 2 wherein the anchor comprises a barb.

4. The apparatus of Embodiment 1 wherein the cannula has a longitudinal axis extending between the proximal and distal ends, and wherein the annulus engaging member is configured to extend from the cannula at an acute angle with respect to the longitudinal axis of the cannula.

5. The apparatus of Embodiment 1 wherein the actuator further comprises a handle having a first position and a second position and being operatively coupled to the annulus engaging member in such a manner that when the handle is moved from the first position to the second position the annulus engaging member engages the annulus.

6. The apparatus of Embodiment 1 wherein the cannula has an inner diameter of approximately 5 mm or less.

7. The apparatus of Embodiment 1 wherein the annulus engaging member is configured to detach from the obturator.

8. The apparatus of Embodiment 1 wherein the annulus engaging member is further adapted to engage at least one suture in such a manner that the suture is drawn through at least a portion of the annulus when the actuator is actuated.

9. An apparatus for performing a surgical operation on an intervertebral disc wherein the disc has an annulus on which an annulotomy may be performed, the apparatus comprising a cannula having a proximal end and a distal end, an obturator coupled to the distal end of the cannula and being adapted for insertion into the annulotomy, means for engaging the annulus operatively coupled to the obturator, and means for actuating operatively coupled to the means for engaging in such a manner that when the means for actuating is actuated the means for engaging engages the annulus thereby securing the apparatus to the annulus.

10. A method of performing a surgical operation on an intervertebral disc wherein the disc has an annulus, the method comprising inserting an obturator into an annulotomy in the annulus, the obturator including a member adapted to engage the annulus and being coupled to a cannula and engaging the annulus with the member by actuating an actuator operatively coupled to the member thereby securing the obturator to the annulus.

11. The method of Embodiment 10 wherein the engaging the annulus with the member further comprises drawing at least one suture through at least a portion of the annulus.

12. The method of Embodiment 10 further comprising leaving the member in the annulus.

It is understood that the present invention can take many forms and embodiments. Accordingly, several variations may be made in the foregoing without departing from the spirit or the scope of the invention.

Having thus described the present invention by reference to certain of its preferred embodiments, it is noted that the embodiments disclosed are illustrative rather than limiting in nature and that a wide range of variations, modifications, changes, and substitutions are contemplated in the foregoing disclosure and, in some instances, some features of the present invention may be employed without a corresponding use of the other features. Many such variations and modifications may be considered obvious and desirable by those skilled in the art based upon a review of the foregoing description of preferred embodiments. Accordingly, it is appropriate that the appended claims be construed broadly and in a manner consistent with the scope of the invention.

We claim:

1. A suture locking device for use in closing an annulotomy of an intervertebral disc wherein the disc has an annulus, the device comprising:
a plate including at least two suture holes wherein each hole is adapted to receive a suture attached to the annulus;
a rotatable actuator disposed adjacent to the plate and adapted to receive the sutures in such a manner that when the actuator rotates the sutures are wound together thereby securing the plate to the annulus; and
a release mechanism operatively coupled to the plate in such a manner that when the release mechanism is actuated the plate is released from the suture locking device.

2. The device of claim 1 wherein the release mechanism further comprises a push button.

3. The device of claim 1 wherein the actuator further comprises a rotatable knob and wherein rotating the knob actuates the actuator.

4. A suture locking device for use in closing a surgical site associated with a mass of tissue, the device comprising:
a suture lock defining a pair of suture holes wherein each hole is adapted to receive a suture attached to the tissue;
a rotatable actuator disposed adjacent to the lock and adapted to receive the sutures in such a manner that when the actuator rotates the sutures are wound together thereby securing the lock to the annulus; and
a release mechanism operatively coupled to the lock in such a manner that when the release mechanism is actuated the device releases the lock.

5. The device of claim 4 wherein the release mechanism further comprises a push button.

6. The device of claim 4 wherein the push button is biased away from the lock.

7. The device of claim 4 wherein the actuator further comprises a rotatable thumbwheel and wherein rotating the thumbwheel actuates the actuator.

8. The device of claim 4 further comprising a hub of the lock wherein the sutures are further wound together around the hub when the lock rotates.

9. The device of claim 4 wherein the lock further comprises a plate adapted to abut the surgical site thereby closing the surgical site.

10. The device of claim 4 further comprising a gear operatively coupled to the actuator and defining a second pair of suture holes wherein each hole is adapted to receive one of the sutures, the gear being operatively coupled to the actuator in such a manner that the gear rotates when the actuator is actuated.

11. The device of claim 10 wherein the gear further comprises a detent, the device further comprising teeth adjacent to the gear and mating with the detent whereby the detent prevents the gear from rotating in one direction.

12. The device of claim 4 further comprising a pull ring connected to a loop, the loop for receiving the sutures and being disposed on the side of the lock and of the actuator opposite the pull ring.

13. The device of claim 4 further comprising an elongate tube, the actuator being disposed at least partially within the elongate tube in such a manner that the device is adapted for closing the surgical site associated with an annulotomy.

14. The device of claim 4 wherein the lock further comprises a plate

15. A suture lock for closing a surgical site associated with a mass of tissue, the lock comprising:
a body defining at least two suture holes wherein each hole is adapted to receive a suture attached to the tissue; and
a hub disposed on a surface of the lock between the suture holes and being adapted to have the sutures wound around the hub thereby securing the lock to the tissue.

16. The lock of claim 15 further comprising a gusset disposed at the end of the hub, the hub being adapted to having the sutures wound around the hub and the gusset being adapted to preventing the sutures from slipping off of the hub as the sutures are wound around the hub.

17. The lock of claim 15 wherein the lock is made of a bio-resorbable material.

18. The lock of claim 15 wherein the body further comprises a plate.

19. An instrument for use in closing a surgical site associated with a mass of tissue, the device comprising:
a proximal end;
a distal end adapted to receive a suture lock defining a pair of suture holes wherein each hole is adapted to receive a suture attached to the tissue;
a rotatable actuator disposed adjacent to the distal end and adapted to receive the sutures in such a manner that when the actuator rotates the sutures are wound together thereby securing the lock to the annulus; and
a release mechanism adapted to be operatively coupled to the lock in such a manner that when the release mechanism is actuated the device releases the lock.

20. A method of closing a surgical site associated with a mass of tissue, the method comprising:
using a suture lock releasably attached to an instrument to receive a pair of sutures attached to the tissue;
drawing the sutures through the lock and through a rotatable member of the instrument, the rotatable member being disposed adjacent to the lock;
rotating the rotatable member whereby the sutures are wound together thereby securing the lock to the tissue; and
releasing the lock from the instrument.

21. The method of claim 20 further comprising cutting the sutures.

22. The method of claim 20 wherein rotating the rotatable member further comprises rotating a thumbwheel of the instrument.

23. The method of claim 20 wherein releasing the lock further comprises pushing a button of the instrument.

24. The method of claim 23 further comprising overcoming a bias of the push button, the bias being away from the lock.

25. The method of claim 20 wherein the drawing the sutures through the lock and the rotatable member further comprises using a pull ring to draw the sutures through an elongate shaft of the instrument.

26. The method of claim 21 wherein the sutures are further wound together around a hub of the lock.
